(19) United States
(12) Patent Application Publication
(10) Pub. No.: US 2011/0150792 A1
(43) Pub. Date: Jun. 23, 2011

(54) ZINC OXIDE AQUEOUS AND NON-AQUEOUS DISPERSIONS

(76) Inventors: Yun Shao, Belle Mead, NJ (US); David Schlossman, Short Hills, NJ (US)

(21) Appl. No.: 12/331,593

(22) Filed: Dec. 10, 2008

(51) Int. Cl. A61K 8/27; A61Q 17/04

(52) U.S. Cl. 424/59

(57) ABSTRACT

Disclosed is a composition that comprises an aqueous or non-aqueous carrier and zinc oxide particles dispersed therein that have a primary particle size of 100 nm or greater with substantially no or few zinc oxide particles having a particle size less than 100 nm.

### BACKGROUND

The present application relates generally to dispersions comprising zinc oxide particles with a primary particle size of 100 nm or greater with substantially no or very few zinc oxide particles with a particle size of less than 100 nm.

Because zinc oxide absorb both UVA and UVB rays of ultraviolet light, they can be used in ointments, creams, and lotions to protect against sunburn and other damage to the skin caused by ultraviolet light. Zinc oxide has been used for centuries for skin protection even though the mechanism was not well understood. Zinc oxide, in particular, is the broadest spectrum UVA and UVB absorber that is approved for use as a sunscreen by the FDA, and is completely photostable.

Zinc oxide, however, has a high refractive index and can cause undesired whitening on skin when its particle size is large. In addition, large particle size can lead to poor skin feel. To improve the aesthetics of suncare products containing zinc oxide, micro zinc oxide was developed and has been used since the 1980s. JP 60-231607 disclosed the use of ZnO with a primary particle size of 10-60 nm. U.S. Pat. No. 5,032,390 disclosed the use of ZnO with a primary particle size of 70-300 nm. These ZnOs are transparent on the skin and aesthetically appealing and are extensively used nowadays. These micro grades typically have primary particle sizes of less than 100 nm when analyzed and are referred to as nano particles.

In light of the perceived health risk associated with nanoparticles, pigment producers have been challenged recently to create particles that are, preferably, all or almost all larger than 100 nm as measured by TEM. Such ZnO was developed and produced by Sumitomo Osaka Cement. The ZnO has a primary particle size in the range of 100-400 nm and almost no zinc oxide particles were found to be under 100 nm according to their report. The particle size is measured by electron microscopy such as TEM or any other size measurement method capable of differentiating particles less than 100 nm. Some variance is possible depending upon the test method used.

The larger sized ZnO particles eliminate the concern over possible skin penetration. Nonetheless, since particles always form hard aggregate, large primary particles can lead to formation of much larger aggregates. Use of the large primary particles in a sunscreen is likely to make the skin appear chalky and unattractive. Moreover, larger particles do not fully provide the degree of protection against ultraviolet light, which is the primary objective of a sunscreen. Therefore, dispersion of the large sized zinc oxide particles and elimination of the large aggregates are necessary.

### SUMMARY OF THE INVENTION

In accordance with the invention methods and compositions are provided for dispersions of larger sized zinc oxide.

As noted above, in the past formulators have been able to achieve the benefits of using nanoparticles in sunscreens with acceptable ultraviolet attenuation and transparency. Many formulators prefer to work with dispersions which are incorporated into end products, which themselves are dispersions of sunscreen attenuating solids. The new larger sized zinc oxide presents unique formulation challenges as their particle size distribution of the zinc oxide powder is wide and the mean size is very large. Stokes law teaches that the dispersion stability is a function of dispersion particle size and viscosity. The large size requires special formulations to promote good dispersion stability because otherwise the large particles will settle out and/or agglomerate. In addition, the large aggregate of these larger particles are much larger than that of micro fine zinc oxides and will generate a rough and very unpleasant skin feel. The need to break apart aggregates and agglomerates to their primary scale and keep them separated using dispersion technology is more important with the new zinc oxide larger particle sizes, in order to obtain a formulation that can be acceptable to consumers.

Applicant has developed a dispersion using the new larger zinc oxide with particles that have particles sizes greater than 100 nm to achieve good dispersion stability and improve their properties to make desirable sunscreens and cosmetics with efficient attenuation of UV light, good transparency, excellent sensory and skin application properties, and reduced skin whitening.

Disclosed herein is a composition comprising an aqueous or non-aqueous carrier and zinc oxide particles dispersed therein, the zinc oxide particles having substantially no particles with a primary particle size of less than 100 nm. The zinc oxide may be present in about 5% to about 80% by weight of the composition. In one embodiment, the zinc oxide particles can be coated with a material such as alumina, silica, an organic material, or combinations thereof. The zinc oxide particle whether coated or uncoated preferably has a primary particle size of 100 nm to about 400 nm.

The carrier may include a dispersing agent, such as an organic dispersant and may be polyhydroxy stearic acid, castor oil phosphate, polyglycerol ester, polyacrylic acid and its salts such as sodium polyacrylate, ammonium polyacrylate, or combinations thereof. In another embodiment, the carrier may include a thickening agent such as an organic polymer gellant or an inorganic thickener that may be a silicon gel, a microcrystalline cellulose, a cellulose derivative, a gelled hydrocarbon, xanthan gum, inorganic clays or organically-modified clays, or combinations thereof. In another embodiment, the carrier may include an oily liquid or a hydrophilic liquid. The oily liquid may be mineral oil, an ester, a silicone fluid, sunflower oil, or combinations thereof. The hydrophilic liquid may be water or mineral water, glycerin, butylene or propylene glycol, caprylyl glycol and other glycols, ethanol, or combinations thereof.

In yet another aspect, disclosed is a composition comprising a carrier comprising an oily liquid or a hydrophilic liquid, a thickening agent, and zinc oxide particles dispersed therein where substantially all the zinc oxide particles have a primary particle size of at least 100 nm or greater. In another embodiment, substantially all the zinc oxide particles have a primary particle size of at least 200 nm or greater.

The zinc oxide may be present in about 5% to about 80% by weight of the composition. In one embodiment, the zinc oxide particles can be coated with a material such as alumina, silica, an organic material, or combinations thereof. The zinc oxide particle whether coated or uncoated preferably has a primary particle size of 100 nm to about 400 nm.

The carrier may include a dispersing agent, such as an organic dispersant and may be polyhydroxy stearic acid, castor oil phosphate, polyglycerol ester, polyacrylic acid and its salts such as sodium polyacrylate, ammonium polyacrylate, or combinations thereof. In another embodiment, the carrier may include a thickening agent such as an organic polymer gellant or an inorganic thickener that may be a silicon gel, a microcrystalline cellulose, a cellulose derivative, a gelled hydrocarbon, xanthan gum, inorganic clays or organically-modified clays, or combinations thereof. In another embodiment, the carrier may include an oily liquid or a hydrophilic liquid. The oily liquid may be mineral oil, an ester, a silicone fluid, sunflower oil, or combinations thereof. The hydrophilic liquid may be water or mineral water, glycerin, butylene or propylene glycol, caprylyl glycol and other glycols, ethanol, or combinations thereof.

In another aspect, any of the various embodiments of the inventive compositions or dispersions described above may be included in cosmetic and/or sunscreen compositions.

### DETAILED DESCRIPTION

The composition disclosed herein is a zinc oxide composition that includes an aqueous or non-aqueous carrier with zinc oxide particles dispersed therein. The zinc oxide particles have a primary particle size of 100 nm or greater with substantially no or few zinc oxide particles having a particle size less than 100 nm. In another embodiment, the zinc oxide particles have a primary particle size of about 120 nm or greater, about 150 nm or greater, or about 200 nm or greater.

The volume weighted mean particle size of the zinc oxide dispersion is less than 0.8 microns with all particle sizes under 3 microns and less than 1% of the particles having a particle size under 100 nm. In another embodiment, the weighted mean particle size is less than 0.4 microns with all particle sizes under 1.5 microns and less than 1% of the particles having a particle size under 100 nm. In another embodiment, there are no particles having a particle size under 100 nm.

The zinc oxide particles may be hydrophobic, for example, by application of a hydrophobic coating on the surface of the zinc oxide particles (core particles), as described in more detail below. The hydrophobic coating may be applied prior to formation of the dispersion, or alternatively in situ, i.e., during dispersion formation. The particles may carry an inorganic coating, separately or in combination with the hydrophobic coating, as described in more detail below. The term “particles of zinc oxide”, as used herein is taken to mean the complete particles, i.e. the core particles plus any coating which has been applied.

The primary particle size of the zinc oxide particles is often representative of the diameter of the particle when the particles are substantially spherical. However, the compositions/dispersions herein also encompass particles of zinc oxides which are non-spherical and in such cases the primary particle size refers to the largest dimension. Non-spherical shapes include, but are not limited to, nodular, acicular, granular, ellipsoidal, hexagonal, prismatic, star-like, flakes, and Y-shaped. The particle size which characterizes the zinc oxides used in the invention is the average size of primary particles, this average size typically being determined by electron microscopy. The size therefore relates to particles of zinc oxide which are not aggregated. Frequently, the primary particles consist of single crystals but may also comprise several crystals fused together.

The zinc oxide composition may be used in cosmetic compositions and sunscreen compositions as a source of zinc oxide particles. The cosmetic composition may be in the form of a liquid, cream, gel, spray-on, or powder sunscreen or make-up. These include products such as foundation or pressed powder, lipstick, blush, eyeshadow, mascara, nail enamel, sunscreen lotions or sprays and so on. Additionally, the cosmetic composition may be anhydrous or water based. Formulators may use the inventive zinc oxide compositions to develop cosmetics or sunscreens having an exceptionally diversified range of formulation or compositional requirements.

Zinc oxide particles in the present composition have a primary particle size of greater than 100 nm, which are substantially free of the safety issues centering on the unsupported, speculative penetration of nanoparticles into those portions of the skin which are not shed in the normal daily epidermis shedding cycle. It is further noted that even in the case of tattoo pigments, which are held in the dermis by complexes of collagen and fibroblasts induced by the scarring effect of the tattooing needle, there appears to be no literature or studies suggesting deleterious effects, despite the use of heavy metal inks, in some cases, compared to the substantially non-reactive pigments used in cosmetics in general and sunscreens in particular.

The zinc oxide particles may have a primary particle size of 100 nm to about 400 nm with substantially none or few of the particles having a particle size under 100 nm. In one embodiment, the zinc oxide particles may have a primary particle size of 100 nm to about 200 nm or 100 nm to about 150 nm with ideally no particles having a particle size under 100 nm. Currently, zinc oxide particles with substantially no particles under 100 nm in particle size as determined by TEM are available from Sumitomo Osaka Cement Company, Limited of Japan as ZnO-C having a mean primary particle size of about 263 nm calculated from its BET specific surface area.

The zinc oxide dispersion composition may include the zinc oxide particles in about 5 to about 80% by weight of the composition. In another embodiment, the zinc oxide particles may be about 30 to about 80% by weight of the composition or about 50 to about 75% by weight of the composition. Finished products using the inventive dispersion as an ingredient will have lower, sometimes substantially lower, concentrations of zinc oxide.

The zinc oxide particles may comprise substantially pure substances or may be coated particles. Methods of coating zinc oxide particles are known to one of skill in the art, as well as various coatings and the amount of coating to be used. The zinc oxide particles may be coated with alumina, silica, an organic material, silicones, or combinations thereof. Other suitable surface treatments may include: phosphate esters (including lecithins), perfluoroalkyl alcohol phosphates, fluorosilanes, isopropyl titanium triisostearate, stearic or other fatty acids, silanes, dimethicone and related silicone polymers or combinations thereof.

For example, zinc oxide particles may be coated with oxides of other elements such as oxides of aluminium, zirconium or silicon, or mixtures thereof such as alumina and silica as disclosed in GB-2205088-A, the teaching of which is incorporated herein by reference. Alternately, such powders may be treated with boron nitride or other known inorganic coatings, singly or in combinations before incorporation into the voids of the particulate. The inorganic coating may be applied using techniques known in the art. A typical process comprises forming an aqueous dispersion of zinc oxide particles in the presence of a soluble salt of the inorganic element whose oxide will form the coating. This dispersion is usually acidic or basic, depending upon the nature of the salt chosen, and precipitation of the inorganic oxide is achieved by adjusting the pH of the dispersion by the addition of acid or alkali, as appropriate. The inorganic coating, if present, is preferably applied as a first layer to the surface of the zinc oxide.

In another embodiment these powders may include an organic coating that gives the pigments hydrophobic properties. The organic coating may be applied to the inorganic coating, if present, or directly to the zinc oxide. The hydrophobic coating agent may be, for example, a silicone, a silane, a metal soap, a titanate, an organic wax, and mixtures thereof. The hydrophobic coating may alternatively include a fatty acid, for example, a fatty acid containing 10 to 20 carbon atoms, such as lauric acid, stearic acid, isostearic acid, and salts of these fatty acids. The fatty acid may be isopropyl titanium trisostearate. With respect to the silicone, the hydrophobic coating may be a methicone, a dimethicone, their copolymers or mixtures thereof. The silicone may also be an organosilicon compound, for example dimethylpolysiloxanes having a backbone of repeating —Me₂SiO— units (“Me” is methyl, CH₃), methyl hydrogen polysiloxanes having a backbone of repeating —MeHSiO— units and alkoxysilanes of formula RₙOSiH(₄₋ₙ) where “R” is alkyl and “n” is the integer 1, 2 or 3. With respect to the silane, the hydrophobic coating agent may be an alkoxysilanes, for example an alkyltriethoxy or an alkyltrimethoxy silanes available from OSI Specialities or PCR. The alkoxysilane may be a triethoxycaprylylsilane or a perfluoroalkylethyl triethoxysilane having a C3 to C12 alkyl group that is straight or branched. One such alkoxysilane is Dynasylan® OCTEO available from Degussa AG. With respect to the metal soap, the hydrophobic coating agent may be a metal myristate, metal stearate, a metal palmitate, a metal laurate or other fatty acid derivatives known to one of skill in the art. The metal, for example, may be magnesium or aluminum. With respect to the titanate, the hydrophobic coating agent may be an organotitanate as taught in U.S. Pat. No. 4,877,604 to Mitchell Schlossman (“Schlossman '604” hereinafter), the disclosure of which is herein incorporated by reference thereto. Schlossman '604 discloses isopropyl titanium triisostearate as one preferred coating agent. With respect to the organic wax, the hydrophobic coating agent may be a synthetic wax like polyethylene or a natural wax like carnauba wax.

If the zinc oxide particles are coated or “surface treated”, the substance forming the coating or treatment may be present in the composition in an amount in a range of about 0.1% to about 35% by weight of the zinc oxide pigment, more preferably 4% to 20%, particularly 6% to 15%, and especially 8% to 12% by weight. For example, alumina may be present in the composition in an amount of about 0.1%-35% by weight, or preferably in about 10% to about 20% by weight of the zinc oxide pigment. In another embodiment, silica may be present in an amount of about 0.1%-35% by weight, or preferably in about 10% to about 20% by weight of the zinc oxide pigment.

The coated zinc oxide particles may also be stabilized using ionic and/or steric stabilization techniques known to one of skill in the art to aid in achieving the stable dispersion of zinc oxide disclosed herein and to prevent the coated particles from agglomerating. Dispersing particles in a liquid is a well-known physical principle.

### Aqueous Steric Stabilizers

Aqueous steric stabilizers that can be included in the dispersion include synthetic polymers and natural thickeners. Exemplary synthetic polymers include, but are not limited to, carbomers, acrylates/C₁₀-C₃₀ alkyl acrylate crosspolymer, acrylates copolymer, polyacrylamide and C₁₃-C₁₄ isoparaffin and laureth-7 (supplied as SEPIGEL® 305 by SEPPIC), acrylamides copolymer and mineral oil and C₁₃-C₁₄ isoparaffin and polysorbate 85 (supplied as SEPIGEL® 501 by SEPPIC), C₁₃-C₁₄ isoparaffin and isostearyl isostearate and sodium polyacrylate and polyacrylamide and polysorbate 60 (supplied as SEPIGEL® 502 by SEPPIC), acrylamide/sodium acryloyldimethyltaurate copolymer and isohexadecane and polysorbate 60 (supplied as Simulgel 600 by SEPPIC), sodium polyacryloyldimethyltaurate and isohexadecane and sorbitan oleate (supplied as Simulgel® 800 by SEPPIC), ammonium polyacrylate and isohexadecane and PEG-40 castor oil (supplied as Simulgel® A by SEPPIC), sodium acrylate/acryloyldimethyltaurate copolymer and isohexadecane and polysorbate 80 (supplied as Simulgel® EG by SEPPIC), sodium acrylate/acryloyldimethyltaurate copolymer and polyisobutene and caprylyl/capryl glucoside (supplied as Simulgel® EG-SL by SEPPIC), hydroxyethyl acrylate/sodium acryloyldimethyltaurate copolymer and squalane and polysorbate 60 (supplied as Simulgel® NS by SEPPIC), hydrophobically modified alkali swellable emulsion polymers (HASE), Dow Corning 190 Surfactant, Dow Corning 193 Surfactant or polyacrylic acid and its salts.

### Organophilic Steric Stabilizers

Organophilic steric stabilizers that can be included in the dispersion include, for example, silicone surfactants such as those sold by Dow Corning under the tradename Dow Corning 5225C Formulation Aid, Momentive 1528 or 1540 Fluid, or Dow Corning Q2-5200, Abil WE97, and the like. Other examples of suitable organophilic steric stabilizers include surfactants sold under the tradename Silwet by Union Carbide, under the tradename Troysol by Troy Corporation, under the tradename Ablusoft by Taiwan Surfactant Co., under the tradename Arkophob by Hoechst.

Other dispersion ingredients may include silicon based surfactants or emulsifiers and organics such as polyhydroxystearic acid, or other viscosity reducers or any other compatible medium component, coating or additive.

### Aqueous and Non-Aqueous Carriers

The zinc oxide particles are dispersed in an aqueous or non-aqueous carrier, which may be an aqueous vehicle, a (volatile or non-volatile) oil-based, hydrocarbon-based or silicone based vehicle, or combination of the same, such as an (oil or silicone)-in-water, water-in-(oil or silicone) formulations. The oil-based liquid may be true oil, such as a vegetable oil, or a mineral oil, an ester, sunflower oil, combinations thereof, or other similar liquids known to one of skill in the art.

Volatile solvents suitable in the composition of the invention include volatile low viscosity silicone fluids such as: water, ethanol, 2-propanol and cyclic silicones. Volatile linear polydimethylsiloxanes are also suitable and generally have from about 2 to 9 silicon atoms. Cyclic silicones are available from various sources including Dow Corning Corporation and General Electric. Dow Corning silicones are sold under the tradenames Dow Corning 244, 245, 344, 345, and 200 fluids. These fluids comprise octamethylcyclotetrasiloxane, decamethylcyclopentasiloxane, hexamethyldisiloxane, or mixtures thereof.

Also suitable as the volatile solvent component are straight or branched chain hydrocarbons having 8-20 carbon atoms, more preferably 10-16 carbon atoms. Examples of suitable hydrocarbons include decane, dodecane, tetradecane, tridecane, and C₈-C₂₀ isoparaffins. Such paraffinic hydrocarbons are available from EXXON (under the ISOPARS trademark), Chevron-Phillips, and the Permethyl Corporation.

The nonvolatile oil may comprise esters of the formula RCO—OR′ where R and R′ are each independently a C₁-C₂₅, preferably a C₄-C₂₀ straight or branched chain alkyl, alkenyl or alkoxy. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, isononyl isononanoate.

The oils may include natural or naturally derived or modified liquids or liquid waxes such as: lanolin, lanolin derivatives, triisocetyl citrate, C₁₀-C₁₈ triglycerides, caprylic/capric/triglycerides, coconut oil, corn oil, cottonseed oil, fruit oils, linseed oil, olive oil, palm oil, illipe butter, rapeseed oil, soybean oil, sunflower seed oil, walnut oil, wheat germ oil, rice bran oil, glyceryl esters and derivatives there of such as acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, trioctyldodecyl citrate, C12-15 alkyl benzoate. Also suitable as the nonvolatile oil are nonvolatile hydrocarbons such as isoparaffins, hydrogenated polyisobutene, hydrogenated polydecene, mineral oil, squalene, petrolatum, and so on.

Suitable silicones include amodimethicone, bisphenylhexamethicone, caprylyl methicone, dimethicone, dimethicone copolyol, dimethiconol, hexadecyl methicone, hexamethyldisiloxane, methicone, methyl trimethicone, phenyl trimethicone, simethicone, dimethylhydrogensiloxane, stearoxy dimethicone, stearoxytrimethylsilane, vinyldimethicone, cyclomethicones and mixtures thereof. Dimethicone, caprylyl methicone, and methyl trimethicone (TMF 1.5 fluid) are available from Shin-Etsu Chemical Co.

Also suitable as the nonvolatile oil are various fluorinated oils such as fluorinated silicones or perfluoropolyethers. Particularly suitable are fluorosilicones such as trimethylsilyl endcapped fluorosilicone oil, polytrifluoro-propyl-methyl-siloxanes, and the like. The nonvolatile component may comprise mixtures of fluorosilicones and dimethylpolysiloxanes. The nonvolatile component may also comprise perfluoropolyethers.

The carrier may be present in an amount of about 0.5% to about 80% by weight of the composition. The carrier may include a thickener, which can be advantageous for stabilizing the composition, and/or an organic dispersant.

### Thickeners

Thickeners may be organic polymer-based gellants or inorganic thickeners. For example, suitable thickeners include fumed silica, aluminum silicate, aluminum starch octenylsuccinate, bentonite, calcium silicate, cellulose, corn starch, diatomaceous earth, fuller's earth, glyceryl starch, hectorite, hydrated silica, kaolin, magnesium aluminum silicate, magnesium carbonate, magnesium silicate, magnesium trisilicate, montmorillonite, microcrystalline cellulose, rice starch, zinc laurate, zinc myristate, zinc neodecanoate, zinc rosinate, zinc stearate, polyethylene, alumina, attapulgite, kaolin, silica silylate, trimethylated silica, and combinations thereof.

Other examples include a silicon gel, a cellulose derivative, a gelled hydrocarbon, waxes (natural and/or synthetic), or combinations thereof. A commercially available microcrystalline cellulose, Avicel, is available from FMC Corporation. Hydroxyethylcellulose, a cellulose derivative, is commercially available from Hercules, Inc. under the trade name Natrosol®. Suitable non-clay gellants include olefin/styrene copolymers, the Versagel series of thickeners, such as Versagel M, Versagel MC1600 and Versagel MC (available from Penreco), and Gel Base (available from Brooks Industries), and propylene carbonate. For example, isohexadecane, a gelled hydrocarbon, is commercially available from Penreco under the trade name Versagel®. The inorganic or modified inorganic thickener may be a smectite or other clay and can be either natural or synthetically-derived such as bentonite, lithium magnesium sodium silicate, kaolin, Veegums (magnesium aluminum silicate), or the like. Another suitable thickener is organically modified clays such as, Bentone 27 and 38 series, as well as Lucentite or similar modified clays. Natural gums such as, xanthan or guar, are also useful thickeners herein, as well as natural and/or synthetic waxes.

Suitable silicone thickeners may also include cross-linked organosiloxane compounds also known as silicone elastomers. Such elastomers may also have hydrophilic groups such as ethylene oxide or, glyceryl groups, or propylene oxide. Examples of suitable silicone elastomers include Dow Corning 9040, sold by Dow Corning, and various elastomeric silicones sold by Shin-Etsu under the KSG tradename including KSG 15, KSG 16, KSG 19, KSG 21, KSG 710, and so on.

### Dispersant

An organic dispersant may be added to the carrier to help keep the zinc oxide particles dispersed therein. The organic dispersant may be a polyhydroxy stearic acid (PHSA), castor oil phosphate, polyglycerol ester, ethylene, butylene, polyethylene or polybutylene glycol, silicones, siloxanes, polyacrylic acid and its salts such as sodium polyacrylate and ammonium polyacrylate, or combinations thereof, or others known to one of skill in the art. The dispersant may be present in an amount of about 0.1% to about 10% by weight of the composition depending on the dispersion medium, or more.

The larger zinc oxide particles with primary particle sizes of 100 nm can be opaque, in the finished product, which may be at least partially a result of agglomerates of the larger particles being present in the dispersion, in the case of sunscreens, this may be to a degree that is not acceptable after formulation of the zinc oxide composition into finished sunscreens and cosmetics. To remove the agglomerates, the zinc oxide particles may be milled or ground in a grinder prior to formulation into a carrier in accordance with the invention so the composition and ultimately the sunscreens and cosmetics made with the composition can have acceptable aesthetics. This can be achieved by adding a small portion of the thickener, for example 2-3%, to the zinc oxide before grinding or at grinding. The remaining substances making up the carrier and dispersed materials and additives are added to the composition and are mixed in a high-speed mixer. The composition may then be milled using a media mill or a ball mill.

The zinc oxide dispersion includes zinc oxide particles with substantially no particles having a particle size below 100 nm. The composition may include the following substances in the ranges given as percent by weight of the composition as shown in Table 1 and Table 2.

TABLE 1

| Substance | % by weight |
|---|---|
| Zinc oxide | 5-80% |
| Alumina (optional) | 0.1-35% |
| Silica (optional) | 0.1-35% |
| An oily or hydrophilic | 0-80% |
| carrier | |
| Dispersant | 0-10% |
| Thickener | 0.5%-35% |

TABLE 2

| Substance | % by weight |
|---|---|
| Zinc Oxide | 5-80% |
| Alumina (optional) | 0.1-35% |
| Silica (optional) | 0.1-35% |
| Water | 0-80% |
| Dispersant | 0-10% |
| Thickener | 0.5%-35% |

The zinc oxide dispersion may also include at least one biocompatible excipient (e.g., buffer (neutralizer or pH adjusters), emulsifier, surfactant, diluent, adjuvant, preservative, and/or electrolyte).

Neutralizers and pH adjusters can be included in the dispersion to stabilize the zinc oxide. Suitable neutralizers and pH adjusters include, but are not limited to, triethanolamine, aminomethyl propanol, ammonium hydroxide, sodium hydroxide, other alkali hydroxides, borates, phosphates, pyrophosphates, cocamine, oleamine, diisopropanolamine, diisopropylamine, dodecylamine, PEG-15 cocamine, morpholine, tetrakis(hydroxypropyl)ethylenediamine, triamylamine, triethanolamine, triethylamine, tromethamine (2-Amino-2-Hydroxymethyl-1,3-propanediol, ascorbic acid and salts thereof, sorbic acid and salts thereof, phosphoric acid and salts thereof, citric acid and salts thereof, lactic acid and salts thereof, glycolic acid and salts thereof, boric acid and salts thereof, acetic acid and salts thereof, and the like, and mixtures thereof.

Preservatives can be included in dispersion. For example, polymethoxy bicyclic oxazolidine, methylparaben, propylparaben, ethylparaben, butylparaben, benzoic acid and the salts of benzoic acid, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzoisothiazolinone, triclosan, sorbic acid, potassium sorbate, benzyl alcohol and salicylic acid salts, and the like.

The present invention is more particularly described in the following non-limiting examples, which are intended to be illustrative only, as numerous modifications and variations therein will be apparent to those skilled in the art.

### Example 1

A dispersion of ZnO-C, available from Sumitomo Osaka Cement, was prepared. One (1) kg of ZnO-C was treated with 40 g of triethoxysilane to render the powder hydrophobic. The powder was then dispersed in a mixture of 940 g of C₁₂-C₁₅ alkyl benzoate and 60 g of polyhydroxystearic acid in a mixer until uniform. The dispersion was milled on a bead mill until none (0%) of the particles had a particle size of less than 100 nm.

TABLE 3
Comparison of particle size

| | Mean size | 100% of particles at | |
|---|---|---|---|
| | (μm) | or less than (μm) | % <100 nm |
| ZnO—C powder | 6.8 | 30 | 0 |
| Dispersion | 0.34 | 1.5 | 0 |

The particle size was measured using a light scattering particle size analyzer.

Other specific exemplary dispersions containing ZnO particles of a specified primary particle are given below in Examples 1-10. The alumina and silica if present are typically coated on the ZnO particles. The dispersions included a carrier, a dispersant, and a thickener. Each substance is listed as a percent by weight of the composition.

### Example 2

| Ingredient | % by wt |
|---|---|
| ZnO—C | 45% |
| Silica | 10% |
| Caprylic/Capric Triglyceride | 41% |
| PHSA | 2% |
| Methicone | 2% |

### Example 3

| Ingredient | % by wt |
|---|---|
| ZnO—C | 60% |
| Alumina | 10% |
| Isononyl Isononanoate | 25% |
| Polyglyceryl-6 Polyricinoleate | 2% |
| Isopropyl Titanium Triisostearate | 3% |

### Example 4

| Ingredient | % by wt |
|---|---|
| ZnO—C | 45% |
| Alumina | 5% |
| Silica | 5% |
| Castor Seed Oil | 43% |
| PHSA | 1% |
| Isopropyl Titanium Triisostearate | 1% |

### Example 5

| Ingredient | % by wt |
|---|---|
| ZnO—C | 40% |
| Alumina | 15% |
| Jojoba Seed Oil | 40% |
| PHSA | 3% |
| Jojoba Esters | 2% |

### Example 6

| Ingredient | % by wt |
|---|---|
| ZnO—C | 35% |
| Silica | 15% |
| Ethyl Trisiloxane | 28% |
| Cyclopentasiloxane | 15% |
| Methicone | 4% |
| Lauryl PEG-9 Polydimethyl siloxyethyl | 3% |

### Example 7

| Ingredient | % by wt |
|---|---|
| ZnO—C | 45% |
| Alumina | 3% |
| Silica | 3% |
| Cyclopentasiloxane | 44% |
| PEG-10 Dimethicone | 2% |
| Methicone | 3% |

### Example 8

| Ingredient | % by wt |
|---|---|
| ZnO—C | 25% |
| Isododecane | 70% |
| PHSA | 3% |
| Methicone | 2% |

### Example 9

| Ingredient | % by wt |
|---|---|
| ZnO—C | 40% |
| Alumina | 15% |
| Deionized Water | 42% |
| Ammonium Polyacylate | 3% |

### Example 10

| Ingredient | % by wt |
|---|---|
| ZnO—C | 35% |
| Silica | 5% |
| Deionized Water | 40% |
| Butylene Glycol | 15% |
| Microcrystalline Cellulose & Cellulose Gum | 5% |

### Example 11

| Ingredient | % by wt |
|---|---|
| ZnO—C | 45% |
| Alumina | 3% |
| Silica | 3% |
| Deionized Water | 44% |
| PEG-10 Dimethicone | 2% |
| Microcrystalline Cellulose & Cellulose Gum | 3% |

### Example 12

| Ingredient | % by wt |
|---|---|
| ZnO—C | 70% |
| Octyldodecyl Neopentanoate | 27% |
| Polyglyceryl-6 Polyricinoleate | 2% |
| Isopropyl Titanium Triisostearate | 1% |

To make the dispersions of Examples 1-10, the carrier was placed in a container with the dispersant and a small portion of the thickener, for example 2-3%, which were mixed together until uniform. Then, the ZnO was added with mixing until the dispersion was mixed well. The mixing speed was increased so high shear can be applied, which took about 10 to about 30 min.

This pre-mix was then ground on a mill. The time for milling depended on the type of machine. However, the end point was determined by the mean size of the aggregate. The end point was reached when the mean size did not reduce further after about 20 to about 30 min. For example, after 30 minutes a pre-mix had a measured mean particle size of 200 nm, milling was continued for an additional 30 minutes, such that after 1 hour total the mean particle size was 190 nm. The change of only 10 nm in the mean particle size after the additional 30 minutes was small enough to consider the mean size of the aggregate to be essentially stable or “not further reduced.” In contrast, if after the additional 30 minutes the mean particle size was reduced to 170 nm, milling was continued for another 30 minutes. The mean particle size was measured with a light scattering size analyzer. Alternately, the mean particle size may be measured with a Dynamic light scattering size analyzer or any suitable instrument. As described above, particle size may also be determined by TEM.

Any of the dispersion disclosed above may be incorporated into other products, such as cosmetic compositions, for example liquid or dry make-ups such as foundation or pressed powder, lipsticks, blushes, eyeshadow, mascara, and sunscreens, paints, coatings, plastics, and other industries where zinc oxides may be useful as will be apparent to those skilled in the art.

### Example 13

A foundation make-up was made as follows:

| Ingredient | % by wt |
|---|---|
| Ethylhexyl Isononanoate | 10.00 |
| Quaternium-18 Hectorite | 2.00 |
| Ethyl Alcohol | 1.00 |
| Polyglyceryl-4 Isostearate & Cetyl PEG/PGG-10/1 | 7.50 |
| Dimethicone (and) Hexyl Laurate | |
| Cetyl Dimethicone | 0.50 |
| Titanium Dioxide (Kobo Products BTD-TTS2)¹ | 3.50 |
| ZnO—C (coated)² | 33.00 |
| Iron Oxide (Kobo Products - WE55Y)³ | 1.00 |
| Iron Oxide (Kobo Products - WE70R)⁴ | 0.42 |
| Iron Oxide (Kobo Products - WE70B)⁵ | 0.20 |
| Sorbitan Isostearate | 0.75 |
| Water | 15.00 |
| 1,2-Hexandiol (and) 1,2-Octanediol | 1.00 |
| Butylene Glycol | 2.50 |
| Sodium Chloride | 1.00 |
| Microcrystalline Wax | 1.00 |
| Carnauba Wax | 1.65 |
| Cyclopentasiloxane (and) C30-45 AlkylCetearyl | 17.23 |
| Dimethicone Crosspolymer | |
| Phenoxyethanol & Methylparaben/Propylparaben & | 0.75 |
| Ethylparaben | |

¹Titanium Dioxide (and) Isopropyl Titanium Triisostearate/Triethoxycaprylylsilane Crosspolymer
²Zinc Oxide (and) C12-15 Alkyl Benzoate (and) Caprylyl Methicone (and) Alumina (and) Polyglyceryl-6 Polyricinoleate (and) Isopropyl Titanium Triisostearate (and) Alumina (and) Silica (Kobo Products)
³Iron Oxide (C.I. 77491) (and) Polyglyceryl-4 Isostearate (and) Cetyl PEG/PGG-10/1 Dimethicone (and) Hexyl Laurate (and) Isopropyl Titanium Triisostearate
⁴Iron Oxide (C.I. 77492) (and) Polyglyceryl-4 Isostearate (and) Cetyl PEG/PGG-10/1 Dimethicone (and) Hexyl Laurate (and) Isopropyl Titanium Triisostearate
⁵Iron Oxide (C.I. 77499) (and) Polyglyceryl-4 Isostearate (and) Cetyl PEG/PGG-10/1 Dimethicone (and) Hexyl Laurate (and)Isopropyl Titanium Triisostearate

To formulate the foundation make up the quaternium-18 hectorite was slowly added to the ethylhexyl isononanoate in container 1 with stirring using a Cowles Dissolver for 20 minutes. The ethyl alcohol was then added with stirring for an additional 20 minutes. Separately in container 2, Polyglyceryl-4 Isostearate & Cetyl PEG/PGG-10/1 Dimethicone (and) Hexyl Laurate, Cetyl Dimethicone, ZnO 100 nm, and the three iron oxide ingredients were stirred together with the Cowles Dissolver for five minutes. Then, the contents of container 1 were slowly added to the contents of container 2 with stirring for five minutes. Next, the sorbitan isostearate was added with additional mixing for 10 minutes. Thereafter, container 2 was heated to 60-65° C. with stirring for an hour. The Cowles Dissolver was removed and mixing was continued with a homogenizer. The microcrystalline wax and the carnauba wax were added at 65° C. with continued homogenization.

In container 3 the water, 1,2-Hexandiol (and) 1,2-Octanediol, Butylene Glycol, and sodium chloride are combined and stirred until clear. Thereafter, the clear contents of container 3 were slowly added to container 2 with continued homogenization while heating to 82° C. with the container cover. Once 82° C. was reached mixing continued for five minutes. Then, the contents were allowed to begin to air cool while homogenization continued. Once the contents reached 70° C. the Cyclopentasiloxane (and) C30-45 AlkylCetearyl Dimethicone Crosspolymer was added. Upon further air-cooling, at 55° C. Phenoxyethanol & Methylparaben/Propylparaben & Ethylparaben was added. Homogenization continued until the composition cooled to 25-30° C.

### Example 14

A sunscreen was made as follows:

| Ingredient | % by wt |
|---|---|
| Ethylhexyl Isononanoate | 10.00 |
| Quaternium-18 Hectorite | 2.00 |
| Ethyl Alcohol | 1.00 |
| Polyglyceryl-4 Isostearate & Cetyl PEG/PGG-10/1 | 7.50 |
| Dimethicone (and) Hexyl Laurate | |
| Cetyl Dimethicone | 0.50 |
| ZnO—C (coated)¹ | 38.12 |
| Sorbitan Isostearate | 0.75 |
| Water | 15.00 |
| 1,2-Hexandiol (and) 1,2-Octanediol | 1.00 |
| Butylene Glycol | 2.50 |
| Sodium Chloride | 1.00 |
| Microcrystalline Wax | 1.00 |
| Carnauba Wax | 1.65 |
| Cyclopentasiloxane (and) C30-45 AlkylCetearyl | 17.23 |
| Dimethicone Crosspolymer | |
| Phenoxyethanol & Methylparaben/Propylparaben & | 0.75 |
| Ethylparaben | |

¹Zinc Oxide (and) C12-15 Alkyl Benzoate (and) Caprylyl Methicone (and) Silica (and) Polyglyceryl-6 Polyricinoleate (and) Isopropyl Titanium Triisostearate (Kobo Products)

The sunscreen composition was formulated following the same procedure for the foundation make-up, except that container 2 includes only Polyglyceryl-4 Isostearate & Cetyl PEG/PGG-10/1 Dimethicone (and) Hexyl Laurate, Cetyl Dimethicone, and ZnO 100 nm.

### Example 15

A sunscreen was made as follows:

| Ingredient | % by wt |
|---|---|
| Zinc Oxide (and) Octyldodecyl Neopentanoate (and) | 31.30 |
| Polyglyceryl-6 Polyricinoleate (and) Isopropyl | |
| Titanium Triisostearate ¹ | |
| Trioctyldodecyl Citrate (and) Titanium Dioxide (and) | 5.63 |
| Triethoxycaprylylsilane (and) Aluminum Hydroxide (and) | |
| Zirconium Dioxide (and) Polyhydroxystearic Acid ² | |
| Cyclopentasiloxane | 5.27 |
| Polyglyceryl-4 Isostearate & Cetyl PEG/PGG-10/1 | 5.00 |
| Dimethicone (and) Hexyl Laurate | |
| Caprylyl Methicone | 3.00 |
| Dimethicone | 1.00 |
| Lithium Magnesium Sodium Silicate (and) | 1.00 |
| Distearyldimonium Chloride | |
| Sorbitan Isostearate | 0.75 |
| Propylene Carbonate | 0.10 |
| Water | 39.57 |
| PEG-150/Decyl Alcohol/SMDI Copolymer | 3.50 |
| Sodium Chloride | 1.00 |
| Propylene Glycol (and) Diazolidinyl Urea | 1.00 |
| (and)Methylparaben (and)Propylparaben | |
| Polysorbate 20 | 0.50 |
| Cyclopentasiloxane (and) Iron Oxides (C.I. 77492) | 1.20 |
| (and) Lauryl PEG-9 Polydimethylsiloxyethyl | |
| Dimethicone (and) Dimethicone/Methicone Copolymer | |
| (and) PEG/PPG-18/18 Dimethicone ³ | |
| Cyclopentasiloxane (and) Iron Oxides (C.I. 77491) | 0.16 |
| (and) Lauryl PEG-9 Polydimethylsiloxyethyl | |
| Dimethicone (and) Dimethicone/Methicone Copolymer | |
| (and) PEG/PPG-18/18 Dimethicone ⁴ | |
| Iron Oxides (C.I. 77499) (and) Cyclopentasiloxane | 0.02 |
| (and) PEG/PPG-20/15 Dimethicone (and) | |
| Triethoxycaprylylsilane ⁵ | |

^{1,} and ² prepared dispersions of a metal oxide available from Kobo Products
^{3, 4,} and ⁵ are color dispersions available from Kobo products

To formulate the sunscreen the zinc oxide dispersion, titanium dioxide dispersion, and the three color dispersions containing the iron oxides were slowly added to and mixed with the cyclopentasiloxane, polyglyceryl-4 isostearate & cetyl PEG/PGG-10/1 dimethicone (and) hexyl laurate, caprylyl methicone, dimethicone, and sorbitan isostearate. Then the lithium magnesium sodium silicate (and) distearyldimonium chloride was added with mixing followed by mixing at high speed for 15 minutes. Next, the propylene carbonate was added with mixing.

The aqueous phase was prepared in a separate container. The aqueous phase includes the water, PEG-150/decyl alcohol/SMDI copolymer, sodium chloride, propylene glycol (and) diazolidinyl urea (and) methylparaben (and) propylparaben, and polysorbate 20. This aqueous phase was slowly added to the mixture above containing the dispersions with mixing at high speed while heating. Heat was removed and metal oxide mixing continued as the mixture cooled to 25-30° C.

While illustrative embodiments have been described above, it is, of course, understood that various modifications will be apparent to those of ordinary skill in the art. Many such modifications are contemplated as being within the spirit and scope of the following claims.

What is claimed is:

1. A composition comprising:
an aqueous or non-aqueous carrier; and
zinc oxide particles dispersed therein, the zinc oxide particles having substantially no particles with a primary particle size of less than 100 nm as measured by transmission electron microscopy.

2. The composition of claim 1 wherein the zinc oxide is present in about 5% to about 80% by weight of the composition.

3. The composition of claim 1 wherein the zinc oxide particles are coated with a material selected from the group consisting of aluminum, silica, an organic material, and combinations thereof.

4. The composition of claim 1 wherein the zinc oxide particles are nodular, acicular, granular, spherical, ellipsoidal, hexagonal, prismatic, star-like, flakes, or Y-shaped.

5. The composition of claim 1 wherein the primary particle size is 100 nm to about 400 nm.

6. The composition of claim 5 wherein the primary particle size is about 120 nm to about 400 nm.

7. The composition of claim 6 wherein the primary particle size is about 150 nm to about 400 nm.

8. The composition of claim 1 wherein the carrier includes a dispersing agent.

9. The composition of claim 8 wherein the dispersing agent is an organic dispersant.

10. The composition of claim 9 wherein the organic dispersant is selected from the group consisting of polyhydroxy stearic acid, castor oil phosphate, polyglycerol ester, polyacrylic acid and its salts such as sodium polyacrylate, ammonium polyacrylate, and combinations thereof.

11. The composition of claim 1 wherein the carrier includes a thickening agent.

12. The composition of claim 11 wherein the thickening agent is an organic polymer gellant or an inorganic thickener.

13. The composition of claim 12 wherein the organic polymer gellant is selected from the group consisting of a silicon gel, a microcrystalline cellulose, a cellulose derivative, a gelled hydrocarbon, xanthan gum, inorganic clays or organically-modified clays, and combinations thereof.

14. The composition of claim 1 wherein the carrier includes an oily liquid or a hydrophilic liquid.

15. The composition of claim 14 wherein the oily liquid is selected from the group consisting of mineral oil, an ester, a silicone fluid, sunflower oil, and combinations thereof.

16. The composition of claim 14 wherein the hydrophilic liquid is selected from the group consisting of water or mineral water, glycerin, butylene or propylene glycol, caprylyl glycol and other glycols, ethanol, and combinations thereof.

17. The composition of claim 1 wherein the composition is included in a cosmetic composition.

18. The composition of claim 1 wherein the composition is included in a sunscreen.

19. A composition comprising:
an oily liquid or a hydrophilic liquid;
a thickening agent; and
zinc oxide particles dispersed therein, wherein substantially all the zinc oxide particles have a primary particle size of at least 100 nm as measured by transmission electron microscopy.

20. The composition of claim 19 wherein substantially all the zinc oxide particles have a primary particle size of at least 200 nm.

21. The composition of claim 19 wherein the zinc oxide is present in about 5% to about 80% by weight of the composition.

22. The composition of claim 19 wherein the zinc oxide particles are coated with a material selected from the group consisting of alumina, silica, an organic material, and combinations thereof.

23. The composition of claim 19 wherein the zinc oxide particles are nodular, acicular, granular, spherical, ellipsoidal, hexagonal, prismatic, star-like, flakes, or Y-shaped.

24. The composition of claim 19 wherein the thickening agent is an organic polymer gellant or an inorganic thickener.

25. The composition of claim 24 wherein the organic polymer gellant is selected from the group consisting of a silicon gel, a microcrystalline cellulose, a cellulose derivative, a gelled hydrocarbon, xanthan gum, inorganic clays or organically-modified clays, and combinations thereof.

26. The composition of claim 19 wherein the oily liquid is selected from the group consisting of mineral oil, an ester, a silicone fluid, sunflower oil, and combinations thereof.

27. The composition of claim 19 wherein the hydrophilic liquid is selected from the group consisting of water or mineral water, glycerin, butylene or propylene glycol, caprylyl glycol and other glycols, ethanol, and combinations thereof.

28. The composition of claim 19 further comprising a dispersing agent.

29. The composition of claim 28 wherein the dispersing agent is an organic dispersant selected from the group consisting of a poly hydroxy stearic acid, a castor oil phosphate, a polyglycerol ester, polyacrylic acid and its salts such as sodium polyacrylate and ammonium polyacrylate, and combinations thereof.

30. The composition of claim 19 wherein the composition is a cosmetic composition.

31. The composition of claim 19 wherein the composition is a sunscreen.
